(19) United States
(12) Patent Application Publication
(10) Pub. No.: US 2026/0268494 A1
(43) Pub. Date: Sep. 10, 2026

(54) SYSTEMS AND METHODS FOR ADAPTIVE PATIENT MONITORING DURING MEDICAL IMAGING PROCEDURES

(71) Applicant: GE Precision Healthcare LLC, Waukesha, WI (US)

(72) Inventors: Aakashkumar Rayate, Bengaluru (IN); Mouleeswaran Kumar, Bengaluru (IN); Nivedha S, Bengaluru (IN); Karthik Chandramouli, Bengaluru (IN); Khaleel Ahamad Nadaf, Bengaluru (IN)

(21) Appl. No.: 19/070,389

(22) Filed: Mar. 4, 2025

(51) Int. Cl. G06T 7/00; G06T 7/33; G06T 7/557

(52) U.S. Cl. CPC G06T 7/0016; G06T 7/337; G06T 7/557; G06T 2207/10048; G06T 2207/30008; G06T 2207/30201

(57) ABSTRACT

A patient monitoring system includes a camera array positioned around a medical imaging device comprising facial cameras with integrated infrared sensors configured to capture facial images from multiple angles and light detection and ranging (LiDAR) based depth sensors configured to generate three-dimensional body mapping data. A processor initializes the system by activating the cameras and sensors, establishes baseline facial expression and body position data, performs parallel real-time monitoring during a medical imaging scan of facial expressions using facial landmark detection and body movements using skeletal reconstruction, analyzes deviations from baseline data against notification and auto-pause thresholds, generates notifications to a technologist when notification thresholds are exceeded, automatically pauses the scan when auto-pause thresholds are exceeded, confirms return of the patient to an acceptable state, and resumes the scan. The system enables comprehensive patient monitoring while maintaining image quality and patient safety.

### TECHNICAL FIELD

Embodiments of the subject matter disclosed herein relate to medical imaging, and more particularly, to real-time monitoring and analysis of patient state during medical imaging procedures.

### BACKGROUND

Medical imaging procedures, such as magnetic resonance imaging (MRI), computed tomography (CT), and other diagnostic imaging modalities, play a critical role in modern healthcare by enabling non-invasive visualization of internal anatomical structures. The diagnostic value of these imaging procedures depends heavily on the quality of the acquired images, which in turn requires patients to remain completely still during scan acquisition. However, maintaining complete stillness throughout imaging procedures presents significant challenges for both patients and healthcare providers.

Patient movement during medical imaging procedures can result in motion artifacts that degrade image quality, potentially leading to missed diagnoses or the need for repeated scans. These artifacts manifest as blurring, streaking, or other distortions that can render images diagnostically unusable. The impact of motion artifacts is particularly pronounced in procedures requiring high precision, such as brain imaging, tumor visualization, or functional MRI studies, where even subtle movements can compromise diagnostic accuracy. The challenge of maintaining stillness is especially acute for vulnerable patient populations, including children, elderly individuals, and patients with cognitive impairments. These patients may struggle to understand or comply with movement restrictions, leading to increased instances of motion artifacts and repeated scans. Additionally, the enclosed environment of imaging systems, particularly MRI scanners, can trigger anxiety, claustrophobia, or panic responses in patients, further exacerbating movement-related issues.

Current approaches to patient monitoring during medical imaging procedures rely heavily on manual observation by imaging technologists. However, technologists face significant limitations in their ability to detect subtle patient movements or signs of distress, particularly in enclosed imaging environments where direct visual observation is restricted. This challenge is compounded by the need for technologists to simultaneously manage multiple aspects of the imaging procedure, resulting in high cognitive load and potential delays in responding to patient movement or discomfort. The use of sedation to manage patient movement, while effective, introduces additional risks including respiratory depression, prolonged recovery times, and potential adverse reactions to anesthesia. Moreover, sedation significantly increases procedure costs and requires additional medical personnel, making it an suboptimal solution for routine imaging procedures.

Traditional patient monitoring approaches also lack the capability to detect and respond to micro-movements or subtle changes in patient state before they result in significant motion artifacts. This reactive rather than proactive approach often leads to workflow inefficiencies, as issues are typically identified only after image quality has been compromised, necessitating repeat scans and increasing radiation exposure in the case of CT imaging. Furthermore, current monitoring systems do not provide objective, quantifiable measures of patient movement or distress levels, making it difficult to establish consistent thresholds for scan interruption or intervention across different procedures and patient populations. This lack of standardization can result in variable image quality and inconsistent patient care protocols across imaging facilities.

Therefore, there exists a clear need for patient monitoring capabilities that can proactively detect and respond to patient movement and distress during medical imaging procedures. Such capabilities would ideally enable real-time assessment of patient state, provide objective measures for intervention decisions, and ultimately improve both image quality and patient experience while maintaining efficient workflow in medical imaging facilities.

### SUMMARY

The present disclosure at least partially addresses the issues described above. In one embodiment, a method for patient monitoring during medical imaging procedures includes initializing a patient monitoring system by activating facial cameras and body movement sensors, establishing baseline data comprising facial expression state data and body position state data of a patient, performing parallel real-time monitoring during a medical imaging scan of facial expressions using facial landmark detection and body movements using skeletal reconstruction, analyzing deviations from the baseline data against notification thresholds and auto-pause thresholds, generating a notification to a technologist when the notification thresholds are exceeded, automatically pausing the medical imaging scan when the auto-pause thresholds are exceeded, confirming return of the patient to an acceptable state, and resuming the medical imaging scan.

In another embodiment, a patient monitoring system includes a camera array positioned around a medical imaging device comprising a plurality of facial cameras with integrated infrared sensors configured to capture facial images from multiple angles and light detection and ranging (LiDAR) based depth sensors configured to generate three-dimensional body mapping data, a memory storing instructions and baseline data, a display device, and a processor communicably coupled to the camera array, memory, and display device. When executing the instructions, the processor is configured to initialize the patient monitoring system by activating the facial cameras and depth sensors, establish baseline data comprising facial expression state data and body position state data of a patient, perform parallel real-time monitoring during a medical imaging scan of facial expressions using facial landmark detection and body movements using skeletal reconstruction, analyze deviations from the baseline data against notification thresholds and auto-pause thresholds, generate a notification to a technologist via the display device when the notification thresholds are exceeded, automatically pause the medical imaging scan when the auto-pause thresholds are exceeded, confirm return of the patient to an acceptable state, and resume the medical imaging scan.

In yet another embodiment, a method includes initializing a patient monitoring system by determining monitoring thresholds based on scan type and patient characteristics, activating facial cameras and three-dimensional (3D) depth sensors, and verifying camera and sensor coverage. The method further includes establishing baseline data by capturing initial facial images and generating facial landmark data, capturing initial 3D depth sensor data and generating skeletal reconstruction data, and storing baseline facial expression state and baseline body position state. After initiating a medical imaging scan, the method includes performing parallel real-time monitoring during the medical imaging scan by analyzing facial expressions using facial landmark detection and comparison to the baseline facial expression state, and analyzing body movements using skeletal reconstruction and comparison to the baseline body position state. The method also includes comparing monitored data against scan-specific notification thresholds and auto-pause thresholds, generating a technologist notification responsive to the notification thresholds being exceeded, and automatically pausing the medical imaging scan responsive to the auto-pause thresholds being exceeded.

The disclosed patient monitoring system and methods provide several technical advantages over conventional approaches. By utilizing parallel processing of both facial expressions and body movements through specialized camera arrays and sensors, the system enables real-time patient state monitoring without requiring direct physical contact with the patient. The integration of automated threshold-based notifications and scan pausing helps prevent motion artifacts and reduce scan retakes while minimizing radiation exposure in CT imaging. The system's ability to maintain continuous monitoring even in low-light conditions through infrared sensors and during patient positioning changes increases reliability across different scanning protocols and anatomical regions. Additionally, the system's dynamic thresholding capabilities, which adjust based on scan type, protocol parameters, and patient characteristics, enable more precise and context-aware monitoring that can be tailored to specific clinical needs.

The system further provides workflow optimization benefits by reducing technologist cognitive load through automated monitoring and intelligent alert prioritization. The combination of facial expression analysis and body movement detection, processed through parallel channels, enables more nuanced and reliable patient state assessment than either modality alone. This multi-modal approach, coupled with continuous learning capabilities that refine monitoring thresholds based on historical scan data, represents a significant advancement in medical imaging procedure management and patient care.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

The above advantages and other advantages, and features of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

Various aspects of this disclosure may be better understood upon reading the following detailed description and upon reference to the drawings in which:

FIG. 1 is a schematic diagram illustrating components of a patient monitoring system during medical imaging, including facial cameras, depth sensors, and processing modules, in accordance with an embodiment of the present disclosure;

FIG. 2 is a block diagram showing a hardware architecture of a patient monitoring system including processor, memory, and connections to imaging and monitoring components, in accordance with an embodiment of the present disclosure;

FIG. 3 is a flowchart depicting a method for monitoring patient state during medical imaging procedures, in accordance with an embodiment of the present disclosure;

FIG. 4 is a flowchart illustrating a method for initializing a patient monitoring system by determining monitoring thresholds and verifying sensor coverage, in accordance with an embodiment of the present disclosure;

FIG. 5 is a flowchart showing a method for establishing baseline patient state data through facial landmark generation and skeletal reconstruction, in accordance with an embodiment of the present disclosure;

FIG. 6 is a flowchart depicting a method for analyzing facial expressions during medical imaging using facial landmark detection and baseline state comparison, in accordance with an embodiment of the present disclosure;

FIG. 7 is a flowchart illustrating a method for analyzing body movements during medical imaging using skeletal reconstruction and position state analysis, in accordance with an embodiment of the present disclosure;

FIG. 8 is a flowchart showing a method for performing combined analysis of facial expression and body movement data against scan-specific thresholds, in accordance with an embodiment of the present disclosure;

FIG. 9 is a flow diagram depicting a method for generating notifications and implementing scan pauses based on threshold exceedances during patient monitoring, in accordance with an embodiment of the present disclosure; and

FIG. 10 is a flow diagram illustrating a method for confirming return to acceptable patient state and resuming a medical imaging scan following a pause, in accordance with an embodiment of the present disclosure.

### DETAILED DESCRIPTION

The following description relates to systems and methods for automated patient monitoring during medical imaging procedures through parallel processing of facial expressions and body movements. Medical imaging procedures often require patients to remain motionless for extended periods, while technologists must simultaneously monitor patient state, manage scanning protocols, and maintain image quality. Conventional monitoring approaches rely heavily on manual observation or basic motion detection, leading to missed patient movements, unnecessary radiation exposure from repeat scans, and reduced workflow efficiency.

The present disclosure describes technical solutions for patient state monitoring through integration of specialized camera arrays, three-dimensional depth sensors, and parallel processing architectures. In various embodiments, the patient monitoring system employs LiDAR-based body mapping for precise motion detection, infrared-enabled facial cameras for low-light monitoring, and data processing to enable real-time intervention before image quality is compromised. The system addresses technical challenges in medical imaging through automated detection of micro-movements, non-verbal patient distress indicators, and integration with existing DICOM protocols.

The disclosed systems and methods provide several technical advantages through its parallel processing architecture, which enables simultaneous analysis of facial expressions and body movements with independent thresholding systems. The system implements protocol-specific thresholds based on scan type, anatomical region, and patient characteristics, while maintaining real-time processing capabilities for immediate intervention. Additional technical benefits include automated decision support through multi-level alert systems, prevention of motion artifacts through predictive monitoring, and HIPAA-compliant data management.

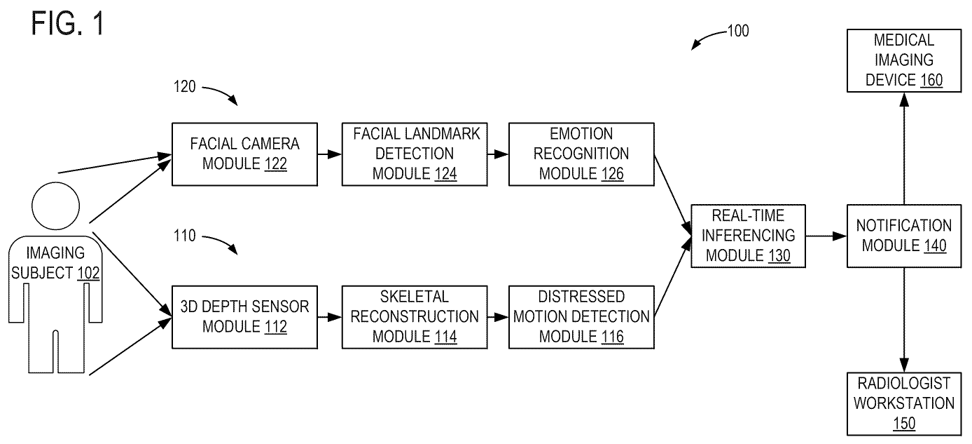

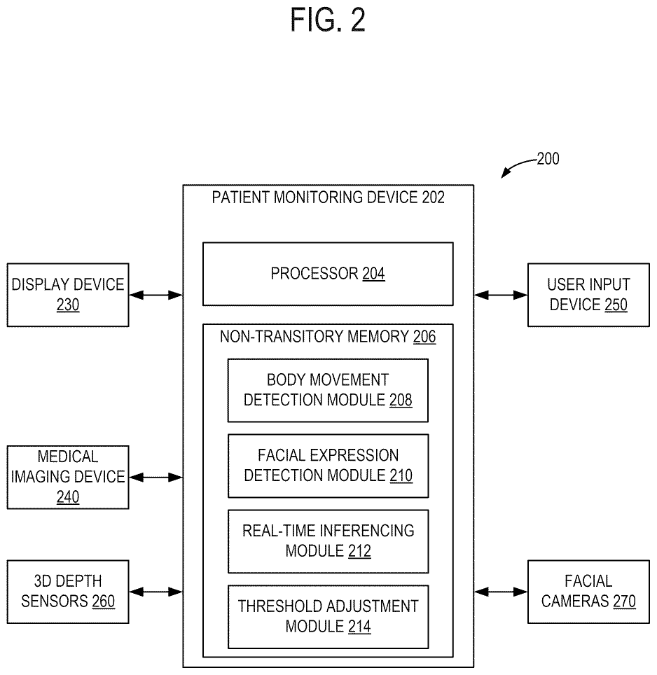

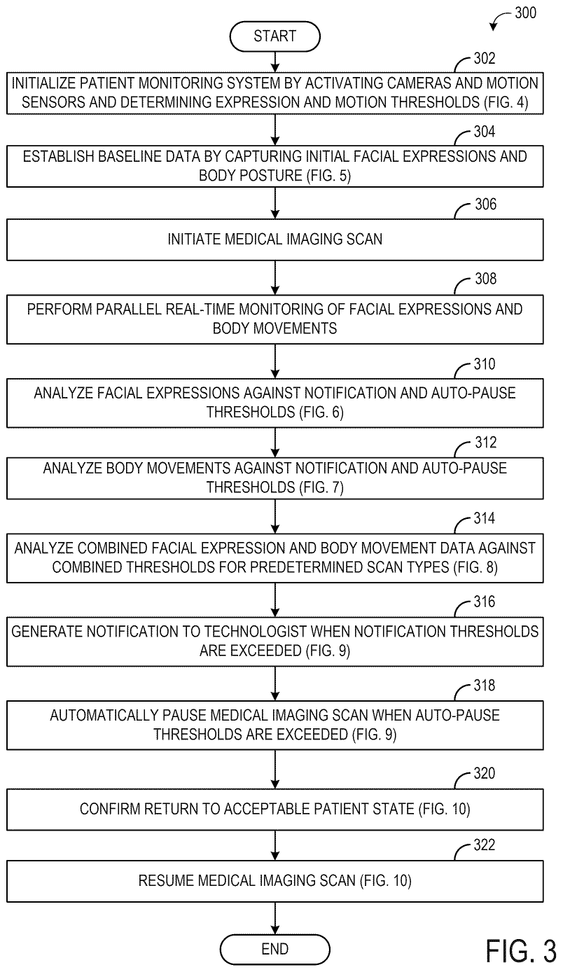

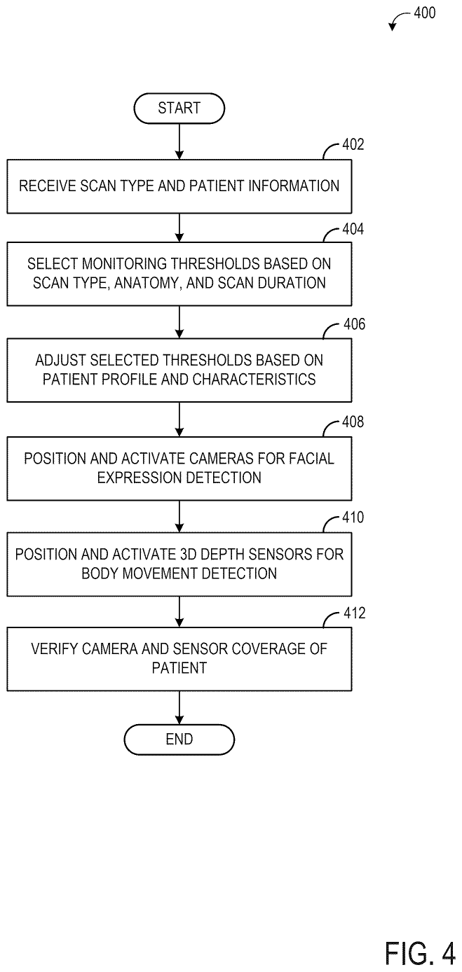

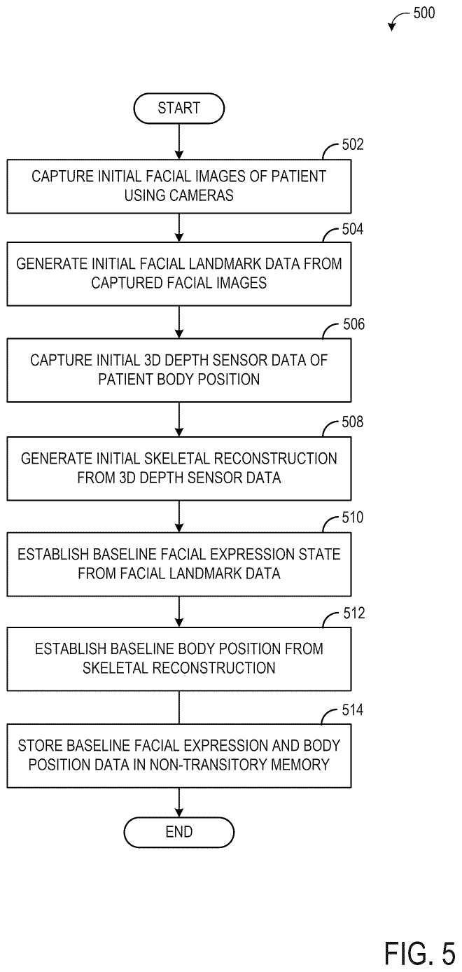

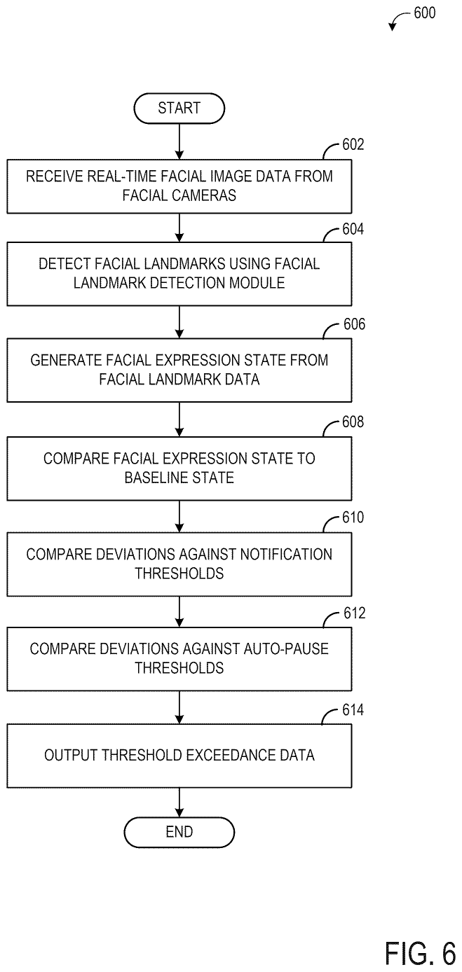

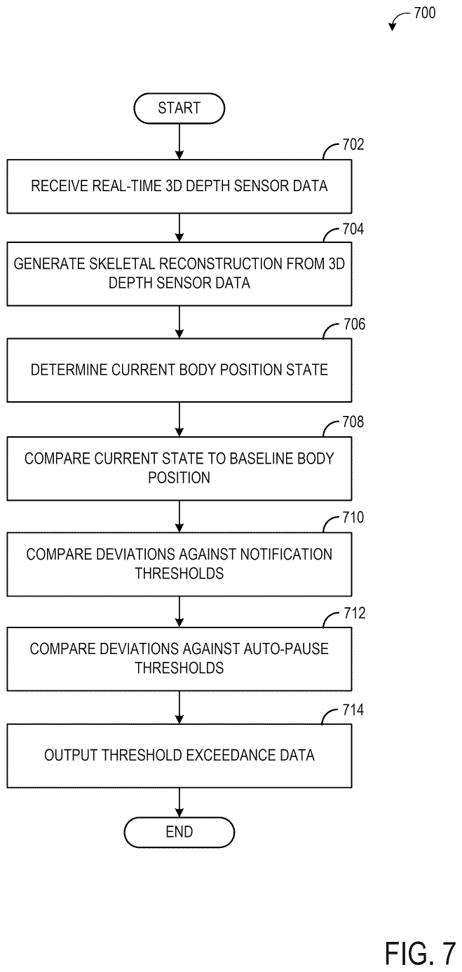

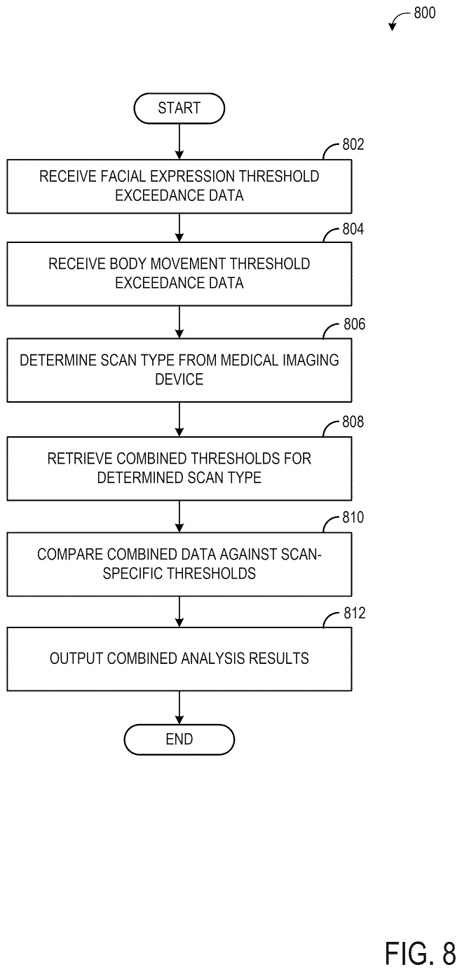

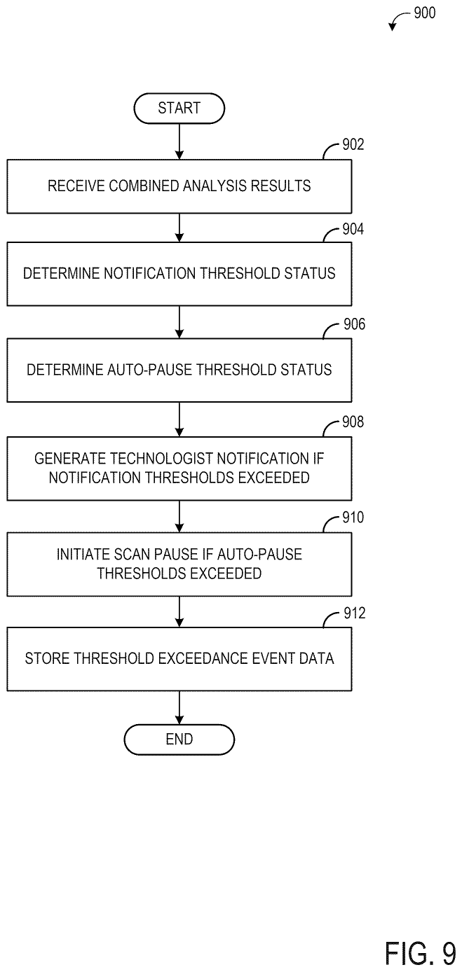

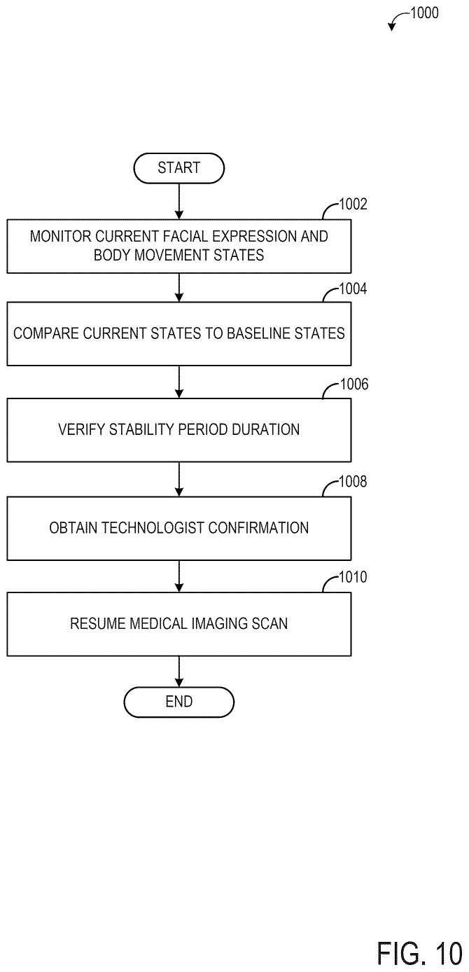

In one embodiment, a patient monitoring system 100, as illustrated in FIG. 1, integrates specialized camera arrays and sensors with medical imaging equipment through a hardware architecture detailed in FIG. 2. The system implements a monitoring method 300 (FIG. 3) that begins with system initialization, including threshold determination and sensor coverage verification (FIG. 4). Baseline patient state data is established through facial landmark generation and skeletal reconstruction processes (FIG. 5), enabling parallel monitoring streams for facial expressions (FIG. 6) and body movements (FIG. 7). The system performs combined analysis of facial and movement data against scan-specific thresholds (FIG. 8), generating appropriate notifications and implementing automated scan pauses (FIG. 9). Following any pause in scanning, the system confirms return to acceptable patient state before resuming the imaging procedure (FIG. 10), ensuring image quality while maintaining patient safety and comfort.

The technical implementations and methods described herein may be better understood through the following detailed descriptions of the system components and processing methods, with reference to the accompanying figures. While various embodiments are described, it should be understood that they are presented by way of example only, and not limitation. Various modifications and variations of the described embodiments will be apparent to those skilled in the art, and such modifications and variations are intended to fall within the scope of the present disclosure.

Referring to FIG. 1, a block diagram of a patient monitoring system 100 for analyzing patient distress during medical imaging is shown. The patient monitoring system 100 implements parallel processing paths for facial analysis and motion detection to monitor a subject 102 during medical imaging procedures. The patient monitoring system 100 includes a motion analysis path 110 and a facial analysis path 120 that operate in parallel to monitor the subject 102 positioned within a medical imaging device 160, with outputs from both paths being processed by a real-time inferencing module 130 to determine patient state and trigger appropriate responses via a radiologist workstation 150.

The subject 102 represents a patient positioned for medical imaging within the medical imaging device 160. In one embodiment, the subject 102 is positioned on a patient table configured to move with respect to a gantry of the medical imaging device 160. In another embodiment, the subject 102 may be positioned using specialized positioning devices or supports based on the anatomical region being scanned and the type of imaging procedure being performed.

The motion analysis path 110 includes a 3D depth sensor module 112, a skeletal reconstruction module 114, and a distressed motion detection module 116 arranged in series. The 3D depth sensor module 112 comprises light detection and ranging (LiDAR) based depth sensors positioned around the medical imaging device 160 to generate three-dimensional body mapping data of the subject 102. In one embodiment, the depth sensors are configured to capture skeletal position data specific to an anatomical region being scanned. In another embodiment, the depth sensors maintain continuous body position monitoring during the medical imaging scan through real-time data acquisition.

The skeletal reconstruction module 114 processes the 3D depth sensor data to generate a skeletal model of the subject 102. In one embodiment, the skeletal reconstruction module 114 employs the OpenPose AI model to perform skeletal tracking and generate real-time skeletal keypoint data. In another embodiment, the module generates a full skeletal reconstruction showing joint positions and body segment orientations relative to baseline positioning data.

The distressed motion detection module 116 analyzes the skeletal reconstruction data to identify motion patterns indicating patient distress or discomfort. In one embodiment, the module compares current skeletal positions against baseline positions to detect deviations exceeding predetermined thresholds. In another embodiment, the module analyzes motion patterns over time to identify repetitive movements or gradual position shifts that may indicate patient discomfort.

The facial analysis path 120 includes a facial camera module 122, a facial landmark detection module 124, and an emotion recognition module 126 arranged in series. The facial camera module 122 comprises a plurality of facial cameras with integrated infrared sensors configured to capture facial images from multiple angles. In one embodiment, the cameras maintain continuous facial visibility during patient positioning changes through strategic camera placement around the medical imaging device 160. In another embodiment, the infrared sensors enable low-light image capture to ensure reliable facial monitoring in varying lighting conditions.

The facial landmark detection module 124 processes the captured facial images to identify and track key facial features. In one embodiment, the module generates facial landmark coordinates to establish baseline facial expression patterns during an initial baseline establishment period. In another embodiment, the module performs continuous facial landmark tracking to detect changes in expression relative to the baseline state.

The emotion recognition module 126 analyzes the facial landmark data to detect signs of patient distress or discomfort. In one embodiment, the module employs the FER+AI model to classify facial expressions and emotional states. In another embodiment, the module generates emotion confidence scores for different emotional states to enable threshold-based alerting.

The real-time inferencing module 130 performs parallel processing of inputs from both the motion analysis and facial analysis paths to determine overall patient state. In one embodiment, the module implements multi-level thresholds comprising notification thresholds triggered by moderate deviations and auto-pause thresholds triggered by severe deviations. In another embodiment, the module performs combined analysis of facial expression and body movement data against scan-specific thresholds based on the type of scan being performed.

The notification module 140 receives patient state determinations from the real-time inferencing module 130 and generates appropriate alerts and control signals. In one embodiment, the notification module 140 transmits alerts to the radiologist workstation 150 when notification thresholds are exceeded. In another embodiment, the module sends control signals to the medical imaging device 160 to automatically pause scanning when auto-pause thresholds are exceeded.

The radiologist workstation 150 provides the user interface for monitoring and controlling the system 100. In one embodiment, the workstation displays real-time patient state information including motion tracking data, facial expression analysis, and alert status indicators. In another embodiment, the workstation enables manual scan control and threshold adjustment while providing access to historical monitoring data for quality assurance review.

The medical imaging device 160 may comprise various imaging modalities including magnetic resonance imaging (MRI), computed tomography (CT), or other diagnostic imaging systems. In one embodiment, the imaging device 160 includes integrated mounting points for the facial cameras and depth sensors to ensure consistent monitoring coverage. In another embodiment, the imaging device 160 implements standardized protocols for scan interruption and resumption based on patient state data from the monitoring system.

Referring to FIG. 2, a patient monitoring system 200 for detecting and analyzing patient movement and facial expressions during medical procedures is shown. The patient monitoring system 200 includes a patient monitoring device 202 communicatively coupled to a medical imaging device 240, 3D depth sensors 260, facial cameras 270, a user input device 250, and a display device 230. The patient monitoring system 200 enables real-time monitoring of patient state during medical imaging procedures through parallel processing of facial expressions and body movements.

The patient monitoring device 202 includes a processor 204 configured to execute machine readable instructions stored in non-transitory memory 206. The processor 204 may be single core or multi-core, and the programs executed thereon may be configured for parallel processing of facial expression and body movement data streams. In some embodiments, processor 204 may include components distributed across multiple devices for coordinated processing. The processor 204 interfaces with the medical imaging device 240 to enable automated pausing of imaging procedures when patient movement or distress is detected.

The non-transitory memory 206 stores multiple specialized detection and analysis modules. The body movement detection module 208 processes data from 3D depth sensors 260 to perform skeletal reconstruction and track patient movements. In one embodiment, the body movement detection module 208 employs OpenPose AI models for skeletal tracking and motion analysis. In another embodiment, the module uses LiDAR-based depth mapping to generate three-dimensional representations of patient position and movement.

The facial expression detection module 210 analyzes input from facial cameras 270 to detect signs of patient discomfort or distress. In one embodiment, the facial expression detection module 210 utilizes FER+ algorithms to identify emotional states through facial landmark detection. In another embodiment, the module employs infrared-enabled cameras for low-light facial monitoring during medical imaging procedures. The facial expression detection module 210 maintains continuous facial visibility through multiple camera angles positioned around the medical imaging device 240.

The real-time inferencing module 212 performs parallel processing of both facial expression and body movement data to generate combined patient state assessments. In one embodiment, the module compares current facial landmarks and skeletal positions against baseline measurements established at scan initiation. In another embodiment, the module analyzes the frequency and severity of movement events and facial expression changes to determine overall patient comfort levels. The real-time inferencing module 212 interfaces with the medical imaging device 240 to enable automated scan pausing when predetermined thresholds are exceeded.

The threshold adjustment module 214 dynamically modifies notification and auto-pause thresholds based on scan type, anatomical region, and patient characteristics. In one embodiment, the module adjusts sensitivity levels based on required stillness for different imaging protocols. In another embodiment, the module modifies thresholds based on historical patient movement patterns and scan quality metrics stored in non-transitory memory 206. The threshold adjustment module 214 enables customized monitoring parameters for optimal scan quality and patient comfort.

The user input device 250 enables technologist interaction with the patient monitoring system 200 through a graphical interface. In one embodiment, the user input device 250 allows manual adjustment of monitoring thresholds and sensitivity levels. In another embodiment, it enables technologist confirmation for resuming paused scans after patient stabilization. The user input device 250 may comprise a touchscreen, keyboard, mouse, or other input mechanisms for controlling monitoring parameters.

The display device 230 presents real-time monitoring data and alerts to imaging technologists. In one embodiment, the display device 230 shows parallel visualization of facial expression and body movement analysis with color-coded alert levels. In another embodiment, it provides trend analysis of patient state over the duration of imaging procedures. The display device 230 may be integrated into existing radiologist workstations or provided as a separate monitoring interface.

The medical imaging device 240 interfaces with the patient monitoring device 202 to enable automated scan control based on patient state. In one embodiment, the medical imaging device 240 receives pause signals when movement or distress thresholds are exceeded. In another embodiment, it provides scan protocol information to the threshold adjustment module 214 for optimizing monitoring parameters. The medical imaging device 240 may be an MRI, CT, or other imaging modality requiring patient stillness.

The 3D depth sensors 260 provide continuous body position monitoring during medical procedures. In one embodiment, the 3D depth sensors 260 use LiDAR technology to generate precise three-dimensional mapping of patient movement. In another embodiment, they employ multiple depth cameras positioned around the imaging area for coverage. The 3D depth sensors 260 maintain continuous monitoring even during patient positioning changes.

The facial cameras 270 enable multi-angle facial expression monitoring throughout imaging procedures. In one embodiment, the facial cameras 270 include infrared sensors for low-light image capture within imaging equipment enclosures. In another embodiment, they comprise a camera array positioned to maintain continuous facial visibility during patient movement. The facial cameras 270 provide input to the facial expression detection module 210 for real-time emotional state analysis.

Referring to FIG. 3, a flowchart of a method 300 for monitoring and controlling a medical imaging scan based on patient state analysis is shown. The method 300 may be employed by a patient monitoring system to detect patient discomfort and movement during medical imaging procedures, enabling automated intervention to maintain image quality and patient safety.

At operation 302, the patient monitoring system is initialized by activating facial cameras and body movement sensors and determining expression and motion thresholds. In one embodiment, the system activates a plurality of facial cameras with integrated infrared sensors configured to capture facial images from multiple angles, along with LiDAR based depth sensors configured to generate three-dimensional body mapping data. The monitoring thresholds may be dynamically determined based on scan type, protocol parameters, anatomical region being scanned, scan duration, and stored patient characteristics. In another embodiment, the system may adjust selected thresholds based on patient profile and characteristics after selecting initial monitoring thresholds based on scan type, anatomy, and scan duration.

At operation 304, baseline data is established by capturing initial facial expressions and body posture of the patient. In one embodiment, the system captures initial facial images using the cameras and generates initial facial landmark data, while simultaneously capturing initial 3D depth sensor data and generating skeletal reconstruction data. The system may establish a baseline facial expression state from the facial landmark data and a baseline body position from the skeletal reconstruction. In another embodiment, the baseline data is validated by verifying consistent facial expression detection across multiple measurements before storing the validated baseline facial expression state and body position data in non-transitory memory.

At operation 306, the medical imaging scan is initiated after verifying proper system initialization and baseline data establishment. The scan may be initiated automatically once the system confirms acceptable baseline patient state, or manually by a technologist after reviewing the baseline data.

At operation 308, the system performs parallel real-time monitoring of facial expressions and body movements during the medical imaging scan. In one embodiment, this involves simultaneously processing facial landmark data from the facial cameras and skeletal reconstruction data from the body movement sensors through parallel processing channels. The system maintains continuous monitoring even in low-light conditions through the infrared sensors and during patient positioning changes.

At operation 310, the system analyzes facial expressions against notification and auto-pause thresholds. In one embodiment, this involves detecting facial landmarks using a facial landmark detection module and generating a current facial expression state, then comparing this state to the baseline state. The system evaluates deviations against notification thresholds based on frequency of detected variations from the baseline facial expression state, patient-specific factors, and scan protocol requirements. In another embodiment, the system classifies facial expression variations into information level variations indicating neutral states, notification level variations indicating moderate discomfort, and auto-pause level variations indicating severe distress.

At operation 312, the system analyzes body movements against notification and auto-pause thresholds. In one embodiment, this involves receiving real-time 3D depth sensor data, generating skeletal reconstruction from this data, determining current body position state, and comparing this state to the baseline body position. The system evaluates deviations against thresholds based on cumulative movement detections and sustained deviation from the baseline state. In another embodiment, the depth sensors are configured to capture skeletal position data specific to the anatomical region being scanned while maintaining continuous body position monitoring during the medical imaging scan.

At operation 314, the system analyzes combined facial expression and body movement data against combined thresholds for predetermined scan types. In one embodiment, this involves receiving both facial expression and body movement threshold exceedance data, determining scan type from the medical imaging device, retrieving combined thresholds specific to that scan type, and comparing the combined data against these scan-specific thresholds. The system may calculate cumulative deviation scores based on frequency of facial expression variations, magnitude of variations from baseline states, and concurrent body movement variations.

At operation 316, the system generates a notification to the technologist when notification thresholds are exceeded. In one embodiment, this involves classifying monitored data into discrete alert levels comprising information level indicators for neutral states, warning level indicators for moderate deviations, and alert level indicators for severe deviations. The system determines a combined alert status based on both facial expression and body movement data before transmitting alert data to a technologist display interface.

At operation 318, the system automatically pauses the medical imaging scan when auto-pause thresholds are exceeded. In one embodiment, this involves initiating an immediate scan pause if patient movement or discomfort exceeds predefined safety limits requiring immediate intervention. The system may store threshold exceedance event data for later analysis and quality improvement.

At operation 320, the system confirms return of the patient to an acceptable state before allowing scan resumption. In one embodiment, this involves monitoring current facial landmark positions and skeletal keypoint positions, comparing these to baseline positions, and verifying position stability over a predetermined time period. The system may require technologist confirmation via a user interface device before proceeding.

At operation 322, the medical imaging scan is resumed once acceptable patient state is confirmed. In one embodiment, this involves verifying both facial expression and body movement states have returned to within acceptable thresholds of baseline states for a sufficient stability period before enabling scan resumption. The system may store scan monitoring data in a patient monitoring database and update monitoring thresholds based on historical scan data correlations.

Following operation 322, method 300 may end. The method enables real-time patient state monitoring during medical imaging procedures through parallel processing of facial expressions and body movements, with automated intervention capabilities to maintain image quality and patient safety while optimizing workflow efficiency.

Referring to FIG. 4, a flowchart of a method 400 for initializing patient monitoring during a medical imaging procedure is shown. The method 400 may be employed by a patient monitoring system to establish monitoring parameters and verify sensor coverage prior to beginning a medical imaging scan. This initialization ensures appropriate detection sensitivity and monitoring coverage based on specific scan requirements and patient characteristics.

At operation 402, the patient monitoring system receives scan type and patient information. The scan type information may include the imaging modality (e.g., MRI, CT, PET, or other diagnostic imaging type), anatomical region being scanned, scan protocol parameters, and expected scan duration. In some embodiments, the patient information may include demographic data such as age range, mobility status, and known conditions that might affect patient movement or comfort during scanning. Additionally, the patient information may include historical scan data indicating previous movement patterns or comfort levels during similar procedures.

At operation 404, the patient monitoring system selects monitoring thresholds based on the scan type, anatomical region, and scan duration. The monitoring thresholds may include notification thresholds that trigger technologist alerts and auto-pause thresholds that automatically halt the scan when exceeded. In one embodiment, the monitoring thresholds are dynamically determined based on the precision requirements of different scan types, with stricter thresholds applied to high-precision procedures such as brain imaging or tumor visualization. In another embodiment, the thresholds may be adjusted based on scan duration, with graduated threshold levels that become more sensitive as scan time increases to account for patient fatigue.

At operation 406, the patient monitoring system adjusts the selected thresholds based on patient profile and characteristics. The adjustment may account for patient-specific factors that influence movement patterns or comfort levels during scanning. In one embodiment, threshold adjustment includes modifying detection frequency thresholds for facial expression variations and body movement sensitivity based on patient age, medical condition, or cognitive status. For example, thresholds may be adjusted to be more sensitive for pediatric patients or patients with conditions that make maintaining stillness difficult. In another embodiment, the system may adjust auto-pause threshold levels based on historical patient data from previous scanning sessions.

At operation 408, the patient monitoring system positions and activates cameras for facial expression detection. The system may employ a camera array comprising multiple facial cameras with integrated infrared sensors configured to capture facial images from multiple angles. In one embodiment, the cameras are positioned around the medical imaging device to maintain continuous facial visibility during patient positioning changes and throughout the scanning procedure. In another embodiment, the infrared sensors enable reliable facial expression monitoring even in low-light conditions typical during certain imaging procedures.

At operation 410, the patient monitoring system positions and activates 3D depth sensors for body movement detection. The system employs LiDAR based depth sensors configured to generate three-dimensional body mapping data. In one embodiment, the depth sensors are positioned to capture skeletal position data specific to the anatomical region being scanned, enabling precise monitoring of movement in the region of interest. In another embodiment, the depth sensors maintain continuous body position monitoring during the medical imaging scan through real-time skeletal reconstruction and position state analysis.

At operation 412, the patient monitoring system verifies camera and sensor coverage of the patient. This verification ensures monitoring capability before initiating the scan. In one embodiment, the system performs a coverage test by capturing initial facial images and 3D depth sensor data to confirm adequate field of view and detection sensitivity across all monitoring devices. In another embodiment, the system verifies that both facial landmark detection and skeletal reconstruction can be performed successfully with the current sensor positioning, adjusting sensor placement if needed to optimize coverage.

Following operation 412, method 400 may end. The initialization process establishes appropriate monitoring parameters and verifies sensor coverage, enabling effective parallel monitoring of facial expressions and body movements throughout the subsequent medical imaging procedure. The system is then prepared to perform real-time analysis of patient state and generate notifications or implement scan pauses when predetermined thresholds are exceeded.

Referring to FIG. 5, a flowchart of a method 500 for establishing baseline patient facial expression and body position data is shown. Method 500 enables baseline data collection for both facial expressions and body positioning of a patient prior to medical imaging procedures, providing reference data against which subsequent patient state monitoring can be compared during scanning.

At operation 502, the system captures initial facial images of the patient using facial cameras. In one embodiment, the facial cameras comprise a camera array positioned around a medical imaging device, where multiple cameras simultaneously capture facial images from different angles to ensure continuous facial visibility during patient positioning changes. The cameras may be fitted with integrated infrared sensors to enable reliable image capture in low-light conditions common in radiology rooms. In another embodiment, the camera array maintains continuous facial visibility by dynamically selecting optimal camera views as the patient's position changes, ensuring consistent facial monitoring even during initial positioning adjustments.

At operation 504, the system generates initial facial landmark data from the captured facial images. In one embodiment, facial landmark detection is performed using a facial expression recognition algorithm trained to identify specific facial features and emotional states indicating patient comfort or distress. The facial landmark data may include detection of features associated with various emotional states including neutral, happiness, sadness, surprise, fear, disgust, anger, and contempt. In another embodiment, the system processes the facial images through parallel channels to generate facial landmark maps that enable detection of subtle changes in expression during subsequent monitoring.

At operation 506, the system captures initial 3D depth sensor data of the patient body position. In one embodiment, LiDAR based depth sensors positioned around the medical imaging device generate three-dimensional body mapping data specific to the anatomical region being scanned. The depth sensors maintain continuous body position monitoring capability during the medical imaging scan. In another embodiment, the system employs stereo cameras that record the position and movement of the patient's body, creating precise 3D maps for detecting micro-movements that might affect scan quality.

At operation 508, the system generates initial skeletal reconstruction from the 3D depth sensor data. In one embodiment, the skeletal reconstruction is performed using OpenPose, an AI-based skeleton tracking model that analyzes posture and movements of the body in real-time. The system generates a detailed skeletal framework showing key anatomical reference points and joint positions. In another embodiment, the skeletal reconstruction process creates a robust 3D model of the patient's positioning, enabling precise tracking of patient stability and detection of involuntary or excess motion during subsequent monitoring.

At operation 510, the system establishes baseline facial expression state from the facial landmark data. In one embodiment, the system processes the facial landmark data to determine a neutral expression state specific to the patient, accounting for natural variations in facial features and resting expressions. The baseline facial expression state serves as a reference point for detecting subsequent changes that may indicate patient discomfort or distress. In another embodiment, the system establishes multiple baseline parameters for different categories of facial expressions, enabling more nuanced detection of changes in patient comfort levels during scanning.

At operation 512, the system establishes baseline body position from the skeletal reconstruction. In one embodiment, the system analyzes the skeletal reconstruction to determine optimal patient positioning specific to the scan type and anatomical region being examined. The baseline body position includes key reference points and acceptable ranges of movement based on scan-specific requirements. In another embodiment, the system establishes multiple positional parameters that account for different types of potential movement, including respiratory motion and involuntary adjustments, creating a baseline for subsequent movement detection.

At operation 514, the system stores baseline facial expression and body position data in non-transitory memory. In one embodiment, the stored baseline data includes both the raw sensor data and processed landmark and skeletal reconstruction data, enabling subsequent comparative analysis during real-time monitoring. The baseline data is securely stored in compliance with HIPAA requirements while remaining readily accessible for real-time comparison during scan monitoring. In another embodiment, the system stores additional contextual data including scan type, protocol parameters, and patient-specific characteristics alongside the baseline data, enabling more precise threshold adjustments during monitoring.

Method 500 provides several technical advantages for patient monitoring during medical imaging procedures. By establishing baseline data through parallel processing of both facial expressions and body positions, the method enables more accurate detection of patient state changes during scanning. The integration of multiple data streams, including infrared-enabled facial cameras and LiDAR-based depth sensors, ensures reliable baseline data collection even in challenging imaging environment conditions. Furthermore, the method's use of AI-based analysis tools, including FER+ for facial expression analysis and OpenPose for skeletal reconstruction, provides precise and objective baseline measurements that enhance the system's ability to detect subtle changes in patient state during subsequent monitoring. This baseline establishment process directly contributes to improved scan quality and reduced need for repeat scans by enabling early detection of patient movement or discomfort.

Referring to FIG. 6, a flowchart of a method 600 for monitoring facial expressions and generating threshold exceedance data is shown. Method 600 enables real-time monitoring of patient facial expressions during medical imaging procedures to detect signs of discomfort or distress that may affect image quality.

At operation 602, the system receives real-time facial image data from facial cameras positioned around a medical imaging device. In one embodiment, the facial cameras comprise a camera array with integrated infrared sensors configured to capture facial images from multiple angles simultaneously, enabling continuous facial visibility during patient positioning changes. In another embodiment, the infrared sensors enable reliable facial image capture in low-light conditions typical of radiology rooms, while maintaining continuous monitoring capability throughout the imaging procedure.

At operation 604, the system detects facial landmarks using a facial landmark detection module. In one embodiment, the facial landmark detection module employs FER+ algorithms trained to identify specific facial features associated with various emotional states including neutral, happiness, sadness, surprise, fear, disgust, anger, and contempt. In another embodiment, the system processes the facial images through parallel channels to generate facial landmark maps that enable detection of subtle changes in expression during monitoring.

At operation 606, the system generates a facial expression state from the facial landmark data. In one embodiment, the system processes the facial landmark data to determine the current emotional state of the patient based on detected facial features and their spatial relationships. In another embodiment, the facial expression state is classified into discrete categories including neutral states, moderate discomfort indicators, and severe distress indicators based on the detected facial landmarks and their temporal evolution.

At operation 608, the system compares the current facial expression state to the baseline state established during system initialization. In one embodiment, the comparison involves analyzing deviations in key facial landmark positions and relationships relative to the baseline neutral expression recorded before scan initiation. In another embodiment, the system tracks the frequency and duration of detected expression variations from the baseline state over predetermined time intervals.

At operation 610, the system compares detected deviations against notification thresholds. In one embodiment, the notification thresholds are dynamically determined based on scan type, protocol parameters, anatomical region being scanned, scan duration, and stored patient characteristics. In another embodiment, the system implements multi-level thresholds where moderate deviations trigger information-level indicators, while more significant variations generate warning-level notifications to the technologist.

At operation 612, the system compares deviations against auto-pause thresholds. In one embodiment, auto-pause thresholds are set higher than notification thresholds and are triggered by severe or persistent facial expressions indicating significant patient distress. In another embodiment, the system evaluates both the magnitude and frequency of expression deviations, automatically pausing the scan if distress indicators exceed predefined safety limits requiring immediate intervention.

At operation 614, the system outputs threshold exceedance data. In one embodiment, the threshold exceedance data includes classification of detected variations into information-level, warning-level, or alert-level indicators, along with specific deviation metrics for technologist review. In another embodiment, the system generates combined threshold exceedance data incorporating both facial expression analysis and concurrent body movement variations for patient state assessment.

Method 600 provides several technical advantages for patient monitoring during medical imaging procedures. By implementing parallel processing of facial expression data with infrared-enabled cameras, the method enables reliable monitoring even in low-light conditions while maintaining continuous facial visibility through multiple camera angles. The integration of FER+ algorithms for facial landmark detection allows precise identification of patient distress indicators before they significantly impact image quality. The method's multi-level thresholding system, with separate notification and auto-pause triggers, enables graduated responses to patient discomfort while maintaining efficient workflow. Additionally, the method's ability to dynamically adjust thresholds based on scan-specific parameters and patient characteristics provides more precise and context-aware monitoring that can be tailored to specific clinical needs.

Referring to FIG. 7, a flowchart of a method 700 for monitoring body position using 3D depth sensor data is shown. Method 700 enables real-time monitoring of patient movement during medical imaging procedures through analysis of three-dimensional depth sensor data and skeletal reconstruction.

At operation 702, the system receives real-time 3D depth sensor data. In one embodiment, light detection and ranging (LiDAR) based depth sensors positioned around a medical imaging device generate three-dimensional body mapping data specific to an anatomical region being scanned. The depth sensors maintain continuous body position monitoring capability during the medical imaging scan through real-time data acquisition. In another embodiment, the system employs multiple depth cameras positioned around the imaging area to provide coverage, with the depth sensors configured to capture skeletal position data even during patient positioning changes.

At operation 704, the system generates skeletal reconstruction from the 3D depth sensor data. In one embodiment, the skeletal reconstruction is performed using OpenPose, an AI-based skeleton tracking model that analyzes posture and movements of the body in real-time. The system generates a detailed skeletal framework showing key anatomical reference points and joint positions relative to baseline positioning data. In another embodiment, the skeletal reconstruction module processes the depth sensor data to generate a full skeletal model showing joint positions and body segment orientations, enabling precise tracking of patient stability and detection of involuntary or excess motion.

At operation 706, the system determines the current body position state based on the skeletal reconstruction. In one embodiment, the system analyzes the skeletal reconstruction to determine current patient positioning specific to the scan type and anatomical region being examined, including key reference points and acceptable ranges of movement based on scan-specific requirements. In another embodiment, the system establishes multiple positional parameters that account for different types of potential movement, including respiratory motion and involuntary adjustments, creating an assessment of current patient state.

At operation 708, the system compares the current state to the baseline body position established during system initialization. In one embodiment, the system compares current skeletal positions against baseline positions to detect deviations exceeding predetermined thresholds specific to the anatomical region being scanned. In another embodiment, the module analyzes motion patterns over time to identify repetitive movements or gradual position shifts that may indicate patient discomfort or compromised scan quality.

At operation 710, the system compares detected deviations against notification thresholds. In one embodiment, the system evaluates body movement variations against the notification thresholds based on frequency of detected variations from the baseline body position state, patient-specific factors from stored patient characteristics, and scan protocol requirements. In another embodiment, the system calculates cumulative deviation scores based on magnitude of variations from baseline states and duration of movement events to determine whether technologist notification is warranted.

At operation 712, the system compares deviations against auto-pause thresholds. In one embodiment, the system evaluates body movement variations against auto-pause thresholds based on cumulative movement detections, sustained deviation from the baseline body position state, and scan-specific movement sensitivity requirements. In another embodiment, the depth sensors are configured to detect micro-movements that might affect scan quality, with the system analyzing the frequency and severity of movement events to determine whether immediate scan interruption is necessary.

At operation 714, the system outputs threshold exceedance data. In one embodiment, the system stores threshold exceedance event data including movement magnitude, duration, and frequency for later analysis and quality improvement. In another embodiment, the system transmits real-time alerts to a technologist display interface when movement patterns indicate potential patient discomfort or risk to image quality, enabling immediate intervention when needed.

Method 700 provides several technical advantages for patient monitoring during medical imaging procedures. By implementing continuous skeletal tracking through LiDAR-based depth sensors and AI-powered reconstruction, the method enables precise detection of patient movement without requiring physical contact with the patient. The parallel processing of movement data against both notification and auto-pause thresholds allows for graduated response to patient movement, helping prevent motion artifacts while minimizing unnecessary scan interruptions. Additionally, the method's ability to maintain continuous monitoring during patient positioning changes and in varying lighting conditions increases reliability across different scanning protocols and anatomical regions. The integration of OpenPose AI modeling for skeletal tracking provides more accurate and consistent movement detection compared to conventional motion sensors, while the storage of threshold exceedance data enables ongoing refinement of monitoring parameters based on historical scan outcomes.

Referring to FIG. 8, a flowchart of a method 800 for analyzing combined patient movement data during medical imaging is shown. Method 800 enables analysis of both facial expressions and body movements during medical imaging procedures to determine overall patient state and trigger appropriate interventions based on scan-specific requirements.

At operation 802, the system receives facial expression threshold exceedance data from a facial expression detection module. In one embodiment, the facial expression threshold exceedance data includes classifications of facial expression variations detected during the medical imaging scan, including information-level variations indicating neutral patient states, notification-level variations indicating moderate patient discomfort, and auto-pause level variations indicating severe patient distress. The facial expression threshold exceedance data may include frequency and duration of detected expression variations from the baseline facial expression state established prior to scan initiation. In another embodiment, the threshold exceedance data incorporates analysis from FER+ algorithms processing real-time facial landmark data captured by infrared-enabled cameras positioned around the medical imaging device.

At operation 804, the system receives body movement threshold exceedance data from a body movement detection module. In one embodiment, the body movement threshold exceedance data includes analysis of skeletal reconstruction data generated from 3D depth sensors, indicating deviations from the baseline body position established during system initialization. The movement threshold exceedance data may include both magnitude and duration of detected movements, classified according to severity levels based on their potential impact on image quality. In another embodiment, the body movement data is derived from LiDAR-based depth sensors providing continuous three-dimensional body mapping data specific to the anatomical region being scanned.

At operation 806, the system determines the scan type from the medical imaging device. In one embodiment, the system interfaces directly with the medical imaging device to obtain scan protocol information including imaging modality (e.g., MRI, CT, PET), anatomical region being examined, and specific protocol parameters. In another embodiment, the system retrieves scan type information from the imaging module controlling the medical imaging device, including details about required stillness levels and scan duration.

At operation 808, the system retrieves combined thresholds specific to the determined scan type. In one embodiment, the combined thresholds are dynamically determined based on the precision requirements of different scan types, with stricter thresholds applied to high-precision procedures such as brain imaging or tumor visualization. The thresholds may be adjusted based on scan duration, with graduated threshold levels that become more sensitive as scan time increases to account for patient fatigue. For example, in brain imaging procedures requiring sub-millimeter precision, the system may implement notification thresholds triggered by facial expression variations exceeding 15% from baseline state when combined with body movements of more than 0.5 mm, while auto-pause thresholds may be triggered by expression variations exceeding 25% combined with movements over 1 mm. In contrast, for larger anatomical regions like abdominal scans, the system may set broader thresholds allowing expression variations up to 25% and movements up to 2 mm before triggering notifications. In another embodiment, the system accesses stored threshold parameters that consider both the anatomical region being scanned and patient-specific characteristics from stored patient profiles. For example, the system may retrieve pre-configured threshold combinations based on patient age groups, with pediatric protocols implementing more sensitive thresholds that trigger at smaller deviations from baseline states. The system may also adjust thresholds based on patient medical history, such as implementing more stringent movement thresholds for patients with conditions affecting motor control. In yet another embodiment, the threshold parameters may be dynamically adjusted during the scan based on real-time analysis of patient compliance patterns, with the system automatically increasing sensitivity when detecting patterns of micro-movements that could impact image quality. The combined thresholds may also incorporate weighting factors that prioritize either facial expression or body movement data based on the specific requirements of different scanning protocols, such as giving greater weight to body movement data during high-resolution cardiac imaging where precise positioning is critical.

At operation 810, the system compares the combined facial expression and body movement data against the scan-specific thresholds. In one embodiment, the system calculates cumulative deviation scores based on frequency of facial expression variations, magnitude of variations from baseline states, and concurrent body movement variations. For example, during brain imaging procedures, the system may assign higher weights to head movement variations when combined with facial expressions indicating discomfort, implementing a weighted scoring system where head movements exceeding 0.5 mm combined with facial distress indicators contribute more significantly to the cumulative deviation score than body movements in other anatomical regions. The system may implement multi-level thresholds where moderate deviations trigger information-level indicators, while more significant variations generate warning-level notifications or auto-pause triggers. For instance, in pediatric protocols, the system may trigger information-level indicators when facial expression variations exceed 15% from baseline combined with body movements of 1 mm, while warning-level notifications may be generated at 20% facial expression deviation with 1.5 mm movement, and auto-pause triggers activated at 25% deviation with 2 mm movement. In another embodiment, the system evaluates both instantaneous and time-averaged deviations against thresholds calibrated for the specific scan type and anatomical region. For example, during cardiac imaging procedures, the system may analyze instantaneous movement deviations exceeding 1 mm combined with sustained facial expressions indicating anxiety, while also monitoring time-averaged movement patterns over 30-second intervals to detect gradual position shifts that could impact image quality. The system may also implement dynamic threshold adjustments based on scan duration, where longer procedures such as contrast-enhanced MRI scans may have graduated threshold levels that become more sensitive to combined facial expression and movement variations as scan time increases, accounting for potential patient fatigue. Additionally, the system may incorporate weighting factors that prioritize either facial expression or body movement data based on the specific requirements of different scanning protocols, such as giving greater weight to body movement data during high-resolution cardiac imaging where precise positioning is critical.

At operation 812, the system outputs the combined analysis results. In one embodiment, the system transmits alert data to a technologist display interface when combined deviations exceed notification thresholds, while automatically triggering scan pauses when deviations exceed auto-pause thresholds. For example, during brain imaging procedures, the system may generate warning-level notifications when facial expression variations exceed 20% from baseline combined with head movements of 1.5 mm, while auto-pause triggers may be activated at 25% facial expression deviation with 2 mm movement. The output may include classification of detected variations into discrete alert levels comprising information level indicators for neutral states, warning level indicators for moderate deviations, and alert level indicators for severe deviations, with color-coded overlays highlighting affected anatomical regions on the technologist display. In another embodiment, the system generates status reports including trend analysis of patient state over the duration of imaging procedures, with color-coded alert levels indicating severity of detected deviations. For instance, the system may overlay yellow indicators for notification-level movement detection around a patient's knee area during orthopedic imaging, along with popup notifications prompting technologist intervention, while red overlays may indicate auto-pause threshold exceedance requiring immediate attention.

Method 800 provides several technical advantages for patient monitoring during medical imaging procedures. The integration of multiple data streams with dynamic thresholding provides early detection of patient discomfort or movement before image quality is compromised, with graduated threshold levels that become more sensitive as scan time increases to account for patient fatigue. Furthermore, the method's ability to adjust thresholds based on scan type and patient characteristics enables sensitivity levels that balance the need for high-quality images with patient comfort and safety, such as implementing more stringent movement thresholds for high-precision procedures like brain imaging while allowing broader thresholds for larger anatomical regions. This approach to patient state analysis directly contributes to improved scan quality, reduced need for repeat scans, and enhanced workflow efficiency in medical imaging facilities through its automated monitoring capabilities and intelligent alert prioritization system.

Referring to FIG. 9, a flowchart of a method 900 for managing threshold exceedance events during medical scanning is shown. Method 900 enables automated response to patient movement and distress during medical imaging procedures through a multi-level threshold monitoring system.

At operation 902, the system receives combined analysis results from facial expression and body movement monitoring. In one embodiment, the combined analysis results include facial expression threshold exceedance data generated through facial landmark detection and body movement threshold exceedance data generated through skeletal reconstruction analysis. In another embodiment, the system determines scan type from the medical imaging device and retrieves scan-specific combined thresholds before processing the analysis results.

At operation 904, the system determines notification threshold status based on the combined analysis results. In one embodiment, the notification thresholds are dynamically determined based on scan type, protocol parameters, anatomical region being scanned, scan duration, and stored patient characteristics. In another embodiment, the system evaluates facial expression variations against the notification thresholds based on frequency of detected variations from the baseline facial expression state, patient-specific factors, and scan protocol requirements.

At operation 906, the system determines auto-pause threshold status based on the combined analysis results. In one embodiment, the auto-pause thresholds are set higher than notification thresholds and are triggered by severe or persistent facial expressions indicating significant patient distress or body movements exceeding predefined safety limits requiring immediate intervention. In another embodiment, the system evaluates body movement variations against the auto-pause thresholds based on cumulative movement detections and sustained deviation from the baseline body position state.

At operation 908, the system generates a technologist notification if notification thresholds are exceeded. In one embodiment, the system classifies monitored data into discrete alert levels comprising information level indicators for neutral states, warning level indicators for moderate deviations, and alert level indicators for severe deviations. In another embodiment, the system transmits real-time alerts to a technologist display interface when movement patterns or facial expressions indicate potential patient discomfort or risk to image quality.

At operation 910, the system initiates an automatic scan pause if auto-pause thresholds are exceeded. In one embodiment, the system automatically pauses the scan if patient movement or discomfort exceeds predefined safety limits and immediate intervention is needed. In another embodiment, the system may operate in different modes: AUTO mode where the system automatically pauses the scan if thresholds are met, INFO mode where the system only shows alerts without interrupting workflow, or OFF mode where the system is completely disabled.

At operation 912, the system stores threshold exceedance event data. In one embodiment, the system stores details including movement magnitude, duration, frequency, and corresponding facial expression variations for later analysis and quality improvement. In another embodiment, the system uses stored threshold exceedance data to refine and update monitoring thresholds based on historical scan outcomes and feedback from clinicians.

Method 900 provides several technical advantages for medical imaging procedures. By implementing a multi-level thresholding system with separate notification and auto-pause triggers, the method enables graduated responses to patient discomfort while maintaining efficient workflow. The integration of dynamically adjusted thresholds based on scan-specific parameters and patient characteristics provides more precise and context-aware monitoring that can be tailored to specific clinical needs. Additionally, the method's ability to store and analyze threshold exceedance data enables continuous refinement of monitoring parameters based on historical outcomes, leading to improved accuracy in detecting patient distress and movement that could impact image quality.

Referring to FIG. 10, a flowchart of a method 1000 for monitoring and verifying patient stability during medical imaging is shown. Method 1000 may be employed by a patient monitoring system to confirm return of a patient to an acceptable state following detection of excessive movement or distress during a medical imaging scan, before resuming the scan.

At operation 1002, the system monitors current facial expression and body movement states of the patient. In one embodiment, facial cameras with integrated infrared sensors capture real-time facial images from multiple angles, while LiDAR-based depth sensors simultaneously generate three-dimensional body mapping data. The facial landmark detection module 124 processes the facial images using FER+algorithms to detect current emotional states through facial landmark analysis, while the skeletal reconstruction module 114 processes the depth sensor data to determine current body position through OpenPose AI-based skeleton tracking. In another embodiment, the system maintains continuous monitoring capability through parallel processing channels, with infrared sensors enabling reliable facial monitoring even in low-light conditions typical of imaging environments, while the depth sensors track body position specific to the anatomical region being scanned.

At operation 1004, the system compares the current states to baseline states established prior to scan initiation. In one embodiment, the real-time inferencing module 130 evaluates current facial landmark positions against the baseline facial expression state, while simultaneously comparing current skeletal keypoint positions to the baseline body position state stored in non-transitory memory 206. The system analyzes deviations from baseline states using both instantaneous and time-averaged measurements to detect any residual signs of patient discomfort or instability. In another embodiment, the system implements multi-level thresholds where facial expression variations within 15% of baseline combined with body movements under 1 mm indicate acceptable stability, while larger deviations require additional monitoring time.

At operation 1006, the system verifies stability period duration to ensure sustained return to acceptable state. In one embodiment, the threshold adjustment module 214 dynamically determines required stability duration based on scan type, anatomical region being examined, and patient characteristics stored in the patient profile. For example, high-precision procedures like brain imaging may require stability verification over a 30-second period, while larger anatomical regions may require shorter stability periods. In another embodiment, the system monitors cumulative stability scores combining both facial expression and movement data over the verification period, requiring consistent scores within acceptable thresholds before proceeding.

At operation 1008, the system obtains technologist confirmation via the user input device 250 before proceeding with scan resumption. In one embodiment, the notification module 140 presents the technologist with trend analysis of patient state over the stability verification period, including color-coded indicators of facial expression and movement variations relative to baseline states. The system may require explicit technologist acknowledgment that patient stability has been verified and scan resumption is appropriate. In another embodiment, the display device 230 provides visualization of both instantaneous and time-averaged stability metrics, allowing the technologist to make an informed decision about patient readiness for scan continuation.

At operation 1010, the medical imaging scan is resumed once acceptable patient state is confirmed. In one embodiment, the system verifies that both facial expression and body movement states have remained within acceptable thresholds of baseline states throughout the required stability period and technologist confirmation has been received before enabling scan resumption. The system may store scan interruption and stability verification data in a patient monitoring database for quality assurance review and refinement of monitoring thresholds. In another embodiment, the system implements a gradual scan power-up sequence while maintaining heightened sensitivity to patient state variations immediately following scan resumption, enabling rapid response to any recurring signs of patient discomfort or movement.

Following operation 1010, method 1000 may end. The method enables reliable verification of patient stability following scan interruption through monitoring of both facial expressions and body movements, with customizable stability requirements based on scan-specific parameters and patient characteristics. This approach helps ensure optimal image quality while maintaining patient safety and comfort during medical imaging procedures.

The disclosure also provides support for a method, comprising: initializing a patient monitoring system by activating facial cameras and body movement sensors, establishing baseline data comprising facial expression state data and body position state data of a patient, performing parallel real-time monitoring during a medical imaging scan of: facial expressions using facial landmark detection, and body movements using skeletal reconstruction, analyzing deviations from the baseline data against notification thresholds and auto-pause thresholds, generating a notification to a technologist when the notification thresholds are exceeded, automatically pausing the medical imaging scan when the auto-pause thresholds are exceeded, confirming return of the patient to an acceptable state, and resuming the medical imaging scan. In a first example of the method, the facial cameras comprise: a camera array positioned around a medical imaging device, infrared sensors integrated with the camera array for low-light image capture, wherein the camera array is positioned to simultaneously capture facial images from multiple angles during the medical imaging scan, and wherein each camera of the camera array is configured to maintain continuous facial visibility during patient positioning changes. In a second example of the method, optionally including the first example, the body movement sensors comprise: light detection and ranging (LiDAR) based depth sensors positioned to generate three-dimensional body mapping data, wherein the depth sensors are configured to: capture skeletal position data specific to an anatomical region being scanned, and maintain continuous body position monitoring during the medical imaging scan. In a third example of the method, optionally including one or both of the first and second examples, performing parallel real-time monitoring comprises: capturing facial landmark data using the facial cameras, capturing skeletal reconstruction data using the body movement sensors, processing the facial landmark data and skeletal reconstruction data through parallel processing channels, and generating combined patient state data from the processed facial and skeletal data. In a fourth example of the method, optionally including one or more or each of the first through third examples, the notification thresholds and auto-pause thresholds are dynamically determined based on: medical imaging device type, scan protocol parameters, anatomical region being scanned, scan duration, and stored patient characteristics. In a fifth example of the method, optionally including one or more or each of the first through fourth examples, generating the notification comprises: classifying monitored data into discrete alert levels comprising: information level indicators for neutral states, warning level indicators for moderate deviations, and alert level indicators for severe deviations, determining a combined alert status based on both facial expression and body movement data, and transmitting alert data to a technologist display interface. In a sixth example of the method, optionally including one or more or each of the first through fifth examples, confirming return of the patient to the acceptable state comprises: monitoring current facial landmark positions and skeletal keypoint positions, comparing current positions to baseline positions, verifying position stability over a predetermined time period, and receiving technologist confirmation via a user interface device. In a seventh example of the method, optionally including one or more or each of the first through sixth examples, the method further comprises: storing scan monitoring data in a patient monitoring database, tracking scan quality metrics associated with patient movement patterns, and updating monitoring thresholds based on historical scan data correlations.

The disclosure also provides support for a patient monitoring system, comprising: a camera array positioned around a medical imaging device, the camera array comprising: a plurality of facial cameras with integrated infrared sensors configured to capture facial images from multiple angles, and light detection and ranging (LiDaR) based depth sensors configured to generate three-dimensional body mapping data, a memory storing instructions and baseline data, a display device, and a processor communicably coupled to the camera array, memory, and display device, wherein, when executing the instructions, the processor is configured to: initialize the patient monitoring system by activating the facial cameras and depth sensors, establish baseline data comprising facial expression state data and body position state data of a patient, perform parallel real-time monitoring during a medical imaging scan of: facial expressions using facial landmark detection, and body movements using skeletal reconstruction, analyze deviations from the baseline data against notification thresholds and auto-pause thresholds, generate a notification to a technologist via the display device when the notification thresholds are exceeded, automatically pause the medical imaging scan when the auto-pause thresholds are exceeded, confirm return of the patient to an acceptable state, and resume the medical imaging scan. In a first example of the system, the camera array is configured to: maintain continuous facial visibility during patient positioning changes, perform low-light image capture using the infrared sensors, and simultaneously capture facial images from multiple angles during the medical imaging scan. In a second example of the system, optionally including the first example, the LiDAR-based depth sensors are configured to: generate real-time three-dimensional body mapping data, capture skeletal position data specific to an anatomical region being scanned, and maintain continuous body position monitoring during the medical imaging scan. In a third example of the system, optionally including one or both of the first and second examples, to perform parallel real-time monitoring, the processor is configured to: capture facial landmark data using the facial cameras, capture skeletal reconstruction data using the depth sensors, process the facial landmark data and skeletal reconstruction data through parallel processing channels, and generate combined patient state data from the processed facial and skeletal data. In a fourth example of the system, optionally including one or more or each of the first through third examples, the processor is configured to dynamically determine the notification thresholds and auto-pause thresholds based on: medical imaging device type, scan protocol parameters, anatomical region being scanned, scan duration, and stored patient characteristics. In a fifth example of the system, optionally including one or more or each of the first through fourth examples, to generate the notification, the processor is configured to: classify monitored data into discrete alert levels comprising: information level indicators for neutral states, warning level indicators for moderate deviations, and alert level indicators for severe deviations, determine a combined alert status based on both facial expression and body movement data, and transmit alert data to the display device.

The disclosure also provides support for a method, comprising: initializing a patient monitoring system by: determining monitoring thresholds based on scan type and patient characteristics, activating facial cameras and three-dimensional (3D) depth sensors, and verifying camera and sensor coverage, establishing baseline data by: capturing initial facial images and generating facial landmark data, capturing initial 3D depth sensor data and generating skeletal reconstruction data, and storing baseline facial expression state and baseline body position state, initiating a medical imaging scan, performing parallel real-time monitoring during the medical imaging scan by: analyzing facial expressions using facial landmark detection and comparison to the baseline facial expression state, and analyzing body movements using skeletal reconstruction and comparison to the baseline body position state, comparing monitored data against scan-specific notification thresholds and auto-pause thresholds, generating a technologist notification responsive to the notification thresholds being exceeded, and automatically pausing the medical imaging scan responsive to the auto-pause thresholds being exceeded. In a first example of the method, establishing baseline data comprises: detecting facial landmarks from the initial facial images to generate facial landmark coordinates, processing the facial landmark coordinates to: generate the baseline facial expression state, and establish baseline facial expression patterns during a baseline establishment period, validating the baseline establishment by: verifying consistent facial expression detection across multiple measurements, and storing the validated baseline facial expression state. In a second example of the method, optionally including the first example, determining monitoring thresholds comprises: setting detection frequency thresholds for facial expression variations from the baseline facial expression state, establishing monitoring levels comprising: notification threshold levels triggered by a first frequency of facial expression variations, and auto-pause threshold levels triggered by a second, higher frequency of facial expression variations, and adjusting detection sensitivity based on the scan type and patient characteristics. In a third example of the method, optionally including one or both of the first and second examples, comparing monitored data against notification thresholds and auto-pause thresholds comprises: retrieving scan protocol parameters including the scan type, accessing the stored patient characteristics, evaluating facial expression variations against the notification thresholds based on: frequency of detected variations from the baseline facial expression state, patient-specific factors from the patient characteristics, and scan protocol requirements, and evaluating body movement variations against the auto-pause thresholds based on: cumulative movement detections, sustained deviation from the baseline body position state, and scan-specific movement sensitivity requirements. In a fourth example of the method, optionally including one or more or each of the first through third examples, performing parallel real-time monitoring during the medical imaging scan further comprises: detecting facial expression variations from the baseline facial expression state, classifying the facial expression variations into: information level variations indicating neutral patient states, notification level variations indicating moderate patient discomfort, and auto-pause level variations indicating severe patient distress, tracking frequency and duration of the classified facial expression variations over time, and adjusting the notification thresholds and auto-pause thresholds based on: cumulative frequency of detected variations, scan protocol requirements, and patient-specific characteristics, wherein the adjusted thresholds determine when to generate the technologist notification or automatically pause the medical imaging scan. In a fifth example of the method, optionally including one or more or each of the first through fourth examples, analyzing facial expressions against notification and auto-pause thresholds comprises: generating a composite patient state assessment by: analyzing facial landmark data to detect variations from the baseline facial expression state, correlating detected facial expression variations with the baseline body position state, calculating cumulative deviation scores based on: frequency of facial expression variations during pre-determined time intervals, magnitude of variations from the baseline facial expression state and concurrent body movement variations from the baseline body position state, and comparing the cumulative deviation scores against scan-specific combined thresholds to determine whether to: generate the technologist notification, or automatically pause the medical imaging scan.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. A method, comprising:
initializing a patient monitoring system by activating facial cameras and body movement sensors;
establishing baseline data comprising facial expression state data and body position state data of a patient;
performing parallel real-time monitoring during a medical imaging scan of:
facial expressions using facial landmark detection, and
body movements using skeletal reconstruction;
analyzing deviations from the baseline data against notification thresholds and auto-pause thresholds;
generating a notification to a technologist when the notification thresholds are exceeded;
automatically pausing the medical imaging scan when the auto-pause thresholds are exceeded;
confirming return of the patient to an acceptable state; and
resuming the medical imaging scan.

2. The method of claim 1, wherein the facial cameras comprise:
a camera array positioned around a medical imaging device;
infrared sensors integrated with the camera array for low-light image capture;
wherein the camera array is positioned to simultaneously capture facial images from multiple angles during the medical imaging scan; and
wherein each camera of the camera array is configured to maintain continuous facial visibility during patient positioning changes.

3. The method of claim 1, wherein the body movement sensors comprise:
light detection and ranging (LiDAR) based depth sensors positioned to generate three-dimensional body mapping data;
wherein the depth sensors are configured to:
capture skeletal position data specific to an anatomical region being scanned, and
maintain continuous body position monitoring during the medical imaging scan.

4. The method of claim 1, wherein performing parallel real-time monitoring comprises:
capturing facial landmark data using the facial cameras;
capturing skeletal reconstruction data using the body movement sensors;
processing the facial landmark data and skeletal reconstruction data through parallel processing channels; and
generating combined patient state data from the processed facial landmark data and skeletal reconstruction data.

5. The method of claim 1, wherein the notification thresholds and auto-pause thresholds are dynamically determined based on:
medical imaging device type;
scan protocol parameters;
anatomical region being scanned;
scan duration; and
stored patient characteristics.

6. The method of claim 1, wherein generating the notification comprises:
classifying monitored data into discrete alert levels comprising:
information level indicators for neutral states,
warning level indicators for moderate deviations, and
alert level indicators for severe deviations;
determining a combined alert status based on both facial expression and body movement data; and
transmitting alert data to a technologist display interface.

7. The method of claim 1, wherein confirming return of the patient to the acceptable state comprises:
monitoring current facial landmark positions and skeletal keypoint positions;
comparing current positions to baseline positions;
verifying position stability over a predetermined time period; and
receiving technologist confirmation via a user interface device.

8. The method of claim 1, further comprising:
storing scan monitoring data in a patient monitoring database;
tracking scan quality metrics associated with patient movement patterns; and
updating monitoring thresholds based on historical scan data correlations.

9. A patient monitoring system, comprising:
a camera array positioned around a medical imaging device, the camera array comprising:
a plurality of facial cameras with integrated infrared sensors configured to capture facial images from multiple angles; and
light detection and ranging (LiDAR) based depth sensors configured to generate three-dimensional body mapping data;
a memory storing instructions and baseline data;
a display device; and
a processor communicably coupled to the camera array, memory, and display device, wherein, when executing the instructions, the processor is configured to:
initialize the patient monitoring system by activating the plurality of facial cameras and depth sensors;
establish baseline data comprising facial expression state data and body position state data of a patient;
perform parallel real-time monitoring during a medical imaging scan of:
facial expressions using facial landmark detection, and
body movements using skeletal reconstruction;
analyze deviations from the baseline data against notification thresholds and auto-pause thresholds;
generate a notification to a technologist via the display device when the notification thresholds are exceeded;
automatically pause the medical imaging scan when the auto-pause thresholds are exceeded;
confirm return of the patient to an acceptable state; and
resume the medical imaging scan.

10. The patient monitoring system of claim 9, wherein the camera array is configured to:
maintain continuous facial visibility during patient positioning changes;
perform low-light image capture using the integrated infrared sensors; and
simultaneously capture facial images from multiple angles during the medical imaging scan.

11. The patient monitoring system of claim 9, wherein the LiDAR based depth sensors are configured to:
generate real-time three-dimensional body mapping data;
capture skeletal position data specific to an anatomical region being scanned; and
maintain continuous body position monitoring during the medical imaging scan.

12. The patient monitoring system of claim 9, wherein to perform parallel real-time monitoring, the processor is configured to:
capture facial landmark data using the facial cameras;
capture skeletal reconstruction data using the depth sensors;
process the facial landmark data and skeletal reconstruction data through parallel processing channels; and
generate combined patient state data from the processed facial landmark data and skeletal reconstruction data.

13. The patient monitoring system of claim 9, wherein the processor is configured to dynamically determine the notification thresholds and auto-pause thresholds based on:
medical imaging device type;
scan protocol parameters;
anatomical region being scanned;
scan duration; and
stored patient characteristics.

14. The patient monitoring system of claim 9, wherein to generate the notification, the processor is configured to:
classify monitored data into discrete alert levels comprising:
information level indicators for neutral states,
warning level indicators for moderate deviations, and
alert level indicators for severe deviations;
determine a combined alert status based on both facial expression and body movement data; and
transmit alert data to the display device.

15. A method, comprising:
initializing a patient monitoring system by:
determining monitoring thresholds based on scan type and patient characteristics;
activating facial cameras and three-dimensional (3D) depth sensors; and
verifying camera and sensor coverage;
establishing baseline data by:
capturing initial facial images and generating facial landmark data;
capturing initial 3D depth sensor data and generating skeletal reconstruction data, and
storing baseline facial expression state and baseline body position state;
initiating a medical imaging scan;
performing parallel real-time monitoring during the medical imaging scan by:
analyzing facial expressions using facial landmark detection and comparison to the baseline facial expression state, and
analyzing body movements using skeletal reconstruction and comparison to the baseline body position state;
comparing monitored data against scan-specific notification thresholds and auto-pause thresholds;
generating a technologist notification responsive to the notification thresholds being exceeded; and
automatically pausing the medical imaging scan responsive to the auto-pause thresholds being exceeded.

16. The method of claim 15, wherein establishing baseline data comprises:
detecting facial landmarks from the initial facial images to generate facial landmark coordinates;
processing the facial landmark coordinates to:
generate the baseline facial expression state; and
establish baseline facial expression patterns during a baseline establishment period;
validating the baseline establishment by:
verifying consistent facial expression detection across multiple measurements; and
storing the validated baseline facial expression state.

17. The method of claim 15, wherein determining monitoring thresholds comprises:
setting detection frequency thresholds for facial expression variations from the baseline facial expression state;
establishing monitoring levels comprising:
notification threshold levels triggered by a first frequency of facial expression variations, and
auto-pause threshold levels triggered by a second, higher frequency of facial expression variations; and
adjusting detection sensitivity based on the scan type and patient characteristics.

18. The method of claim 15, wherein comparing monitored data against notification thresholds and auto-pause thresholds comprises:
retrieving scan protocol parameters including the scan type;
accessing the stored patient characteristics;
evaluating facial expression variations against the notification thresholds based on:
frequency of detected variations from the baseline facial expression state;
patient-specific factors from the patient characteristics; and
scan protocol requirements; and
evaluating body movement variations against the auto-pause thresholds based on:
cumulative movement detections;
sustained deviation from the baseline body position state; and
scan-specific movement sensitivity requirements.

19. The method of claim 15, wherein performing parallel real-time monitoring during the medical imaging scan further comprises:
detecting facial expression variations from the baseline facial expression state;
classifying the facial expression variations into:
information level variations indicating neutral patient states;
notification level variations indicating moderate patient discomfort; and
auto-pause level variations indicating severe patient distress;
tracking frequency and duration of the classified facial expression variations over time; and
adjusting the notification thresholds and auto-pause thresholds based on:
cumulative frequency of detected variations;
scan protocol requirements; and
patient-specific characteristics;
wherein the adjusted thresholds determine when to generate the technologist notification or automatically pause the medical imaging scan.

20. The method of claim 15, wherein analyzing facial expressions against notification and auto-pause thresholds comprises:
generating a composite patient state assessment by:
analyzing facial landmark data to detect variations from the baseline facial expression state;
correlating detected facial expression variations with the baseline body position state;
calculating cumulative deviation scores based on:
frequency of facial expression variations during pre-determined time intervals;
magnitude of variations from the baseline facial expression state and
concurrent body movement variations from the baseline body position state; and
comparing the cumulative deviation scores against scan-specific combined thresholds to determine whether to:
generate the technologist notification, or
automatically pause the medical imaging scan.
